(19) United States
(12) Patent Application Publication
(10) Pub. No.: US 2015/0265662 A1
(43) Pub. Date: Sep. 24, 2015

(54) LAYERING AND MICROENCAPSULATION OF THERMAL SENSITIVE BIOLOGICALLY ACTIVE MATERIAL USING HEAT ABSORBING MATERIAL LAYERS HAVING INCREASING MELTING POINTS

(71) Applicant: KeepCool Ltd., Tel Aviv (IL)

(72) Inventor: Adel Penhasi, Holon (IL)

(73) Assignee: KEEPCOOL LTD., Tel Aviv (IL)

(21) Appl. No.: 14/357,571

(22) PCT Filed: Nov. 11, 2012
(86) PCT No.: PCT/IL2012/050453

§ 371 Date: May 11, 2014

Related U.S. Application Data
(60) Provisional application No. 61/558,479, filed on Nov. 11, 2011.

(51) Int. Cl. A61K 35/747; A61K 9/50

(52) U.S. Cl. CPC A61K 35/747; A61K 9/5042

(57) ABSTRACT

A layered microencapsulation structure and a method of preparation of the layered structure are provided herein. The layered microcapsules comprises different coating layers having a specific arrangement order where each layer is composed of at least one phase change material which is able to absorb heat from surroundings and still to keep constant temperature or an insignificant increase in temperature via a fusion process occurring at a specific temperature (e.g. melting point) and a core substrate that has a heat-sensitive component which is entrapped therein. The layered microencapsulation structure is designed in such a way that the layers are arranged with increasing order of the melting point from inside to outside. The method of microencapsulation comprises the step of dry cold granulation of a sensitive active material using a melt material resulting in a core substrate and layering using heat absorbing materials having increasing melting points. The core substrate is coated by different layers of phase change material having different melting points resulting in a layered microcapsule structure. After layering process the layered microcapsule may be optionally coated by an outermost layer which is soluble in GI tract.

### FIELD OF THE INVENTION

The present invention is related to probiotics, and particularly but not exclusively to methods and compositions for maintaining probiotic stability during one or more of manufacturing, storing and/or transporting, and administration to a mammalian subject, such as a human subject.

### BACKGROUND OF THE INVENTION

Probiotics are live microbial food supplements which beneficially affect the host by supporting naturally occurring gut flora, by competing harmful microorganisms in the gastrointestinal tract, by assisting useful metabolic processes, and by strengthening the resistance of the host organism against toxic substances. The beneficial effects that probiotics may induce are numerous. Few examples are; the reduction of lactose intolerance, the inhibition of pathogenic bacteria and parasites, the reduction of diarrhea, activity against Helicobacter pylori, the prevention of colon cancer, the improvement or prevention of constipation, the in situ production of vitamins, the modulation of blood fats, and the modulation of host immune functions. In domesticated and aquatic animals they also can improve growth, survival and stress resistance associated with diseases and unfavorable culture conditions. Therefore, there is considerable interest in including probiotics into human foodstuffs and into animal feed.

Probiotic organisms should survive for the lifetime of the product, in order to be effective. Probiotic organisms are usually incorporated into milk products, such as yogurts.

Probiotic organisms are also usually administrated as OTC drug such as Mutaflor, the probiotic drug containing E. Coli strain Nissle 1917 as active ingredient. The need of such probiotics is specially strengthened after an antibiotic treatment during which the natural micro-flora existing in the lower GI tract may be hardly harmed. However, in this case the beneficial microorganisms should be delivered in the lower GI tract and specifically to the colon.

Many medicinal treatments in which administration of antibiotics is involved generally kill all, or most of, the beneficial bacteria in the intestine.

During a course of antibiotics and for an extended period afterward it is mostly recommended to protect the intestine by taking probiotics.

An alternate approach particularly if a bad Candida has developed after an antibiotic treatment is to take a protective supplement including an appropriate probiotic which should be delivered in the lower GI tract. This treatment is supposed to displace Candida and other harmful bacteria.

Either the probiotics are administrated as an OTC drug or a protective supplement it is mostly of interest to provide colon specific delivery of probiotics. For this reason the probiotics should be coated with an appropriate film coating polymer to hinder the release of the probiotics in the upper GI tract for colon-specific delivery.

The activity and long term stability of probiotics bacteria may be affected by a number of environmental factors; for example, temperature, pH, the presence of water/humidity and oxygen or oxidizing or reducing agents. It is well known that many heat sensitive probiotics instantly lose their activity during storage at even ambient temperatures (AT). Generally, Probiotic bacteria must be dried before or during mixing with other foodstuff ingredients. The drying process can often result in a significant loss in activity due to the temperature, mechanical, chemical and osmotic stresses induced by the drying process. Loss of activity may occur at many distinct stages, including drying, during initial manufacturing, final product preparation (including capsulation and coating process if the probiotics are intended for a medicine treatment) (upon exposure to high temperature, high humidity and oxygen), transportation, long term storage and after consumption and passage in the gastrointestinal (GI) track (exposure to low pH, proteolytic enzymes and bile salts). Manufacturing food or feedstuffs with live cell organisms or probiotics is in particular challenging, because the probiotics are very sensitive to oxygen, temperature and moisture which are in fact the conditions of the foodstuff.

Many probiotics exhibit their beneficial effect mainly when they are alive. Hence, they need to survive the manufacturing process and shelf life. Likewise, they should survive the gastro-intestinal tract conditions such as very low pH existing in stomach, upon consumption of the food before reaching their place of colonization. Although many commercial probiotic products are available for animal and human consumptions, most of them lost their viability during the manufacture process, transport, storage and in the animal/human GI tract.

To compensate for such loss, an excessive quantity of probiotics is included in the product in anticipation that a portion will survive and reach their target. In addition to questionable shelf-life viability for these products, such practices are certainly not cost-effective.

Various protective agents have been used in the art, with varying degrees of success. These include proteins, certain polymers, skim milk, glycerol, polysaccharides, oligosaccharides and disaccharides. Disaccharides, such as sucrose and trehalose, are particularly attractive cryoprotectants because they are actually help plants and microbial cells to remain in a state of suspended animation during periods of drought. Trehalose has been shown to be an effective protectant for a variety of biological materials, both in ambient air-drying and freeze-drying.

Alternatively, the probiotic microorganisms can be encapsulated by enteric coating techniques involve applying a film forming substance, usually by spraying liquids containing enteric polymer and generally other additives such as sugars or proteins onto the dry probiotics (Ko and Ping WO 02/058735). However, the enteric coating process is by itself involved with heating and high level of humidity which are both destructive parameters for viability of probiotics.

### BRIEF SUMMARY OF THE INVENTION

Many probiotics may be temperature sensitive and thus suffer from lack of an extended shelf life. Therefore, they need protection during processing, transporting and storage as well as during delivery to the gastro intestinal tract to maintain viability. The background art fails to provide a solution to this problem of maintaining probiotic viability during manufacturing, storage and/or transport and ingestion, while also providing probiotics in a form that is suitable for ingestion by a mammalian subject, such as a human subject for example.

The present invention overcomes these drawbacks of the background art by providing a layered composition for containing the probiotics, in which the layers are temperature specific, comprising materials that are suitable for human ingestion. The term “human” is also assumed to encompass mammals generally according to at least some embodiments of the present invention. Methods of use and of preparation thereof are also provided. For the purpose of discussion only and without any desire to be limited in any way, the composition preferably is prepared in the form of layered microcapsules as described herein.

The layered microcapsules may comprise different coating layers having a specific arrangement order where each layer may be composed of at least one phase change material which is able to absorb heat from surroundings and still to keep constant temperature or an insignificant increase in temperature via a fusion process occurring at a specific temperature (e.g. melting point) and a core substrate that has a heat-sensitive component which is entrapped therein. The layered microencapsulation structure is designed in such a way that the layers are arranged with increasing order of the melting point from inside to outside. Optionally, the composition is then coated with an enteric coating layer.

A non-limiting example of a method of microencapsulation optionally comprises dry cold granulation of a sensitive active material using a melt material resulting in a core substrate and layering using heat absorbing materials having increasing melting points. Optionally, additionally or alternatively, a hot melt process may be used for certain layers, such as for an external enteric coating layer for example.

The core substrate may be coated by different layers of phase change material having different melting points resulting in a layered microcapsule structure. After the layering process, the layered microcapsule may be optionally coated by an enteric coating layer which is soluble in the GI tract.

Without wishing to be limited by a closed list, it was unexpectedly found that probiotic bacteria are protected for an extended period of time at ambient temperature when preserved in a certain protective composition. Additional qualities of the protecting composition are a fast and cost effective preparation process and protection in many kinds of solid dosage forms.

The present invention provides, in at least some embodiments, a process and composition for the preparation of heat resisting probiotic bacteria for a nutritionally or nutraceutically or pharmaceutically acceptable product comprising: (a) a core composition in form of particles containing probiotic bacteria and at least one substrate comprising optionally at least one sugar compound such as maltodextrin, trehalose, lactose, galactose, sucrose, fructose and the like, a stabilizer such as oxygen scavenger (antioxidant) such as L-cysteine base or L-cysteine hydrochloride, at least one binder having a melting point lower than 50° C. and higher than 25° C. preferably lower than 45° C. and higher than 25° C. and most preferably lower than 40° C. and higher than 25° C., optionally a filler such as microcrystalline cellulose, and optionally other food grade ingredients where the total amount of probiotics in the mixture is from about 10% to about 90% by weight of the core composition (b) a first coating layer which is the innermost coating layer comprising at least one first phase change material (PCM) having a melting point lower than 60° C. and higher than 20° C., preferably lower than 55° C. and higher than 20° C. and most preferably lower than 50° C. and higher than 20° C. forming a stable film around the probiotics core particles, (c) a second coating layer comprising at least one second phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the first coating layer, the second PCM has a melting point which is higher than the first PCM, (d) optionally a third coating layer comprising at least one third phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the second coating layer, the third PCM has a melting point which is higher than the second PCM, (e) optionally subsequently more coating layers, where each layer comprises at least one phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the former coating layer, where each PCM has a melting point which is higher than the PCM composing the former layer (beneath layer), (d) optionally and preferably an outermost layer comprising a polymer which is soluble in GI tract, thereby obtaining a layered structure providing stabilized probiotic granules or microencapsules for forming a dosage form for oral administration. Optionally the probiotic containing particles are in the form of a granulate or a finer particulate, such as a powder for example.

Both PCM layers as well as outermost layer may optionally further comprise at least one excipient, such as, for example, a plasticizer, a glidant including but not limited to silicon dioxide, lubricant and anti-adherents, including but not limited to microcrystalline cellulose, talc or titanium dioxide. The stabilized bacteria are capable to resist during manufacturing or preparation process or further handling process such as coating process where there is an exposure to high temperature. The resultant stabilized bacteria are further capable to resist during storage conditions at ambient temperature.

The resultant stabilized probiotic granules or microencapsules are optionally and preferably suitable for admixing/adding to food products such as chocolate, cheese, creams, sauces, mayonnaise and biscuit fill-in, the probiotic particles comprising oxygen, ambient temperatures resistant and humidity resistant probiotic bacteria. The stabilized bacteria are capable to resist during manufacturing or preparation process where there is exposure to high temperature. The stabilized bacteria are further capable to resist during storage conditions at ambient temperature even after they are added to a food product.

### BRIEF DESCRIPTION OF THE DRAWINGS

The invention described herein in accordance with some demonstrative embodiments will be understood and appreciated more fully from the following detailed description taken in conjunction with the drawings in which:

FIGS. 1(a) and 1(b) are schematic illustrations of graphs showing heat content Q as a function of temperature T.

FIG. 2 is a schematic illustration of a graph that demonstrates the effect of slow cooling rate on melting point of PEG with different molecular weights.

FIG. 3 is a schematic illustration of a graph that demonstrates the effect of fast cooling rate on melting point of PEG with different molecular weights.

FIG. 4 is a schematic illustration of a graph that demonstrates the effect of slow cooling on melting point of a blend comprising PEG 1500 and PEG 6000.

FIG. 5 is a schematic illustration of a graph that demonstrates the effect of fast cooling on melting point of a blend comprising PEG 1500 and PEG 6000

FIG. 6 is a schematic illustration of a graph that demonstrates the effect of fast cooling on melting point of a blend comprising PEG 1000 and PEG 6000

FIG. 8 is a schematic illustration of a thermogram of a laminated structure comprising PEG 1000 and PEG 2000

FIG. 9 is a schematic illustration of a thermogram of a laminated structure comprising PEG 1000, PEG 2000 and PEG 4000

FIG. 10 is a schematic illustration of a thermogram of a laminated structure comprising PEG 1000, PEG 2000 and PEG 8000

FIG. 11 is a schematic illustration of a thermogram of a laminated structure comprising PEG 1000, PEG 4000 and PEG 8000

FIG. 12 is a schematic illustration of a thermogram of a laminated structure comprising PEG 1500, PEG 6000 and PEG 8000

### DETAILED DESCRIPTION OF PREFERRED EMBODIMENTS

For many sensitive probiotic bacteria as well as pharmaceutically or nutraceutically active materials temperature maintenance below a critical temperature, at which they may loss a significant part of vitality, viability and/or biological activity, is very important. There are many reasons that such products which are based on sensitive probiotic bacteria as well as pharmaceutically or nutraceutically active materials are exposed may be exposed to increased temperatures, including without limitation increased temperatures during manufacturing, transportation and storage.

It has now been found that probiotic bacteria may be surprisingly efficiently stabilized for use in food preparation and pharmaceutical, nutraceutical and nutritional products preparation process by layering process based on a desired combination of phase change material coating layers having a specific arrangement order. The bacteria were formulated in a core or granule coated with coating layers, thereby obtaining probiotic compositions providing viable probiotic organisms even after a prolonged time of storage at ambient temperature, the composition being further stable on storage and shelf life of the food stuff or pharmaceutical, nutraceutical and nutritional products containing the protected probiotics according to the present invention and capable of administering viable bacteria to the gastrointestinal tracts after the oral administration.

The materials of each layer were selected so that the manufacturing process temperature is lower for layers closer to the core containing the probiotics, but higher away from the core containing the probiotics. Such a combination enables the probiotic to be protected, yet also provides desirable characteristics for the resultant composition, in terms of strength and stability of the overall coated product, ability to use desirable materials on the outer layers which require high temperatures, the ability to use desirable manufacturing processes for the outer layers which require high temperatures and so forth.

The present invention in at least some embodiments is directed to a process for the preparation of protected probiotics against high temperatures for incorporating into foodstuffs such as creams, biscuits creams, biscuit fill-in, chocolates, sauces, cheese, mayonnaise and etc or pharmaceutical, nutraceutical and nutritional products in a solid dosage form such as particles, beads, microspheres, granules, mini-tablets, tablets, caplets, capsules, MUPS, and liquid dosage form such as syrups, beverages and alike.

In an important embodiment of the invention, the dosage form containing stabilized probiotic granules or miroencapsules is further optionally and preferably coated by an enteric polymer which may further provide protection through GI tract destructive parameters such as low pH environments and enzymes.

The outermost layer comprising a polymer which further provides protection against either oxygen or humidity or both oxygen and humidity and which is soluble in GI tract, thereby obtaining a layered structure providing stabilized probiotic granules or microsphere for forming a dosage form for oral administration. In an important embodiment of the invention, the dosage form containing stabilized probiotic granules or microencapsules is further optionally and preferably coated by an enteric polymer which may further provide protection through GI tract destructive parameters such as low pHs and enzymes. The product may also optionally be prepared in a process that comprises a hot melt granulation process, without harming the probiotics.

According to the present invention there is provided a process for the preparation of high temperature resisting probiotic bacteria for providing high stability and prolonged shelf life at ambient temperature for a food product or for a nutritionally or nutraceutically or pharmaceutically acceptable product comprising a process, according to a preferred embodiment, for preparing microencapsules, granular or particular probiotic having i) a core with probiotic bacteria and which may contain at least one stabilizing agent, antioxidant, substrate, filler, binder, and other excipients and further having ii) a first coating layer which is the innermost coating layer comprising at least one first phase change material (PCM) having a melting point lower than 60° C. and higher than 20° C., preferably lower than 55° C. and higher than 20° C. and most preferably lower than 50° C. and higher than 20° C. forming a stable film around the probiotics core particles and further having iii) a second coating layer comprising at least one second phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the first coating layer, the second PCM has a melting point which is higher than the first PCM and further optionally having iv) a third coating layer comprising at least one third phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the second coating layer, the third PCM has a melting point which is higher than the second PCM and further optionally subsequently having v) more coating layers, where each layer comprises at least one phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the former coating layer, where each PCM has a melting point which is higher than the PCM composing the former layer (underlying layer), wherein the first, second, third or any other PCMs, being used in the layering process, can chemically be either similar to or different from each other and further optionally and preferably having vi) an outermost layer comprising a polymer which is soluble in GI tract, thereby obtaining a layered structure providing stabilized probiotic granules or microsphere for forming a dosage form for oral administration.

Each PCM layer as well as outermost layer may optionally further comprise at least one excipient, such as, for example, a plasticizer, a glidant including but not limited to silicon dioxide, lubricant and anti-adherents, including but not limited to microcrystalline cellulose, talc or titanium dioxide. The stabilized bacteria are capable to resist during manufacturing or preparation process or further handling process such as coating process where there is an exposure to high temperature. The stabilized bacteria are further capable to resist during storage conditions at ambient temperature.

According to the present invention there is provided a process for the preparation of high temperature resisting probiotic bacteria for providing high stability and prolonged shelf life at ambient temperature for a healthy food product or for a nutritionally or nutraceutically or pharmaceutically acceptable product comprising a process, according to a preferred embodiment, for preparing microencapsules, granular or particular probiotic having i) a core with probiotic bacteria and which may contain at least one stabilizing agent, antioxidant, substrate, filler, binder, and other excipients and further having ii) a first coating layer which is the innermost coating layer comprising at least one first phase change material (PCM) having a melting point lower than 60° C. and higher than 20° C., preferably lower than 55° C. and higher than 20° C. and most preferably lower than 50° C. and higher than 20° C. forming a stable film around the probiotics core particles and further having iii) a second coating layer comprising at least one second phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the first coating layer, the second PCM has a melting point which is higher than the first PCM and further optionally having iv) a third coating layer comprising at least one third phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the second coating layer, the third PCM has a melting point which is higher than the second PCM and further optionally subsequently having v) more coating layers, where each layer comprises at least one phase change material (PCM) having a melting point lower than 60° C. and higher than 20° C., preferably lower than 55° C. and higher than 20° C. and most preferably lower than 50° C. and higher than 20° C. forming a stable film around the probiotics core particles coated with the former coating layer, where each PCM has a melting point which is higher than the PCM composing the former layer (beneath layer)), wherein the first, second, third or any other PCMs, being used in the layering process, are chemically same but are different from each other by their molecular weights so that the first PCM has the lowest molecular weight and the outermost PCM has the higher molecular weight, and further optionally and preferably having vi) an outermost layer (exterior layer) comprising a polymer which is soluble in GI tract, thereby obtaining a layered structure providing stabilized probiotic granules or microsphere for forming a dosage form for oral administration. Both PCM layers as well as outermost layer may optionally further comprise at least one excipient, such as, for example, a plasticizer, a glidant including but not limited to silicon dioxide, lubricant and anti-adherents, including but not limited to microcrystalline cellulose, talc or titanium dioxide. The stabilized bacteria are capable to resist during manufacturing or preparation process or further handling process such as coating process where there is an exposure to high temperature. The stabilized bacteria are further capable to resist during storage conditions at ambient temperature.

In a preferred embodiment of the invention the probiotic bacteria comprise at least one heat sensitive probiotic bacteria, the stabilized probiotic core granule or core mixing according to the invention is a coated granule, comprising at least two layered phases, for example a core and two coats, or a core and three or more coats. Usually, two of the coats are composed of two PCMs having different melting points, the inner layer has the lowest melting point, contributing mainly to protecting against high temperatures usually ambient temperature, the other coats are more PCM layers which are responsible for protecting against higher temperatures, the other coat is the exterior coating layer which is responsible for preventing transmission of humidity and/or oxygen into the core during the storage and shelf life and/or for protecting against destructive parameters through GI tract such as low pHs and enzymes. Usually, there are two of the layers that contribute maximally to the high temperature resistance, however, the stabilized probiotic granule of the invention may comprise more layers that contribute to the stability process of the bacteria at higher temperatures, as well as to their stability during storing the food, pharmaceutical, nutraceutical or nutritional product and during safe delivery of the bacteria to the intestines. Likewise, the two or PCM layers composing the layered structure of the stabilized probiotic granule or microencapsule may be chemically the same polymers but with different viscosities or molecular weights.

In a preferred process of manufacturing probiotic healthy food product or nutritionally or nutraceutically or pharmaceutically acceptable product, probiotic bacteria is mixed with at least one substrate comprising at least one sugar and/or at least one oligosaccharide or polysaccharides (as a supplemental agent for the bacteria), and optionally other food grade additives such as stabilizers, fillers, binders, antioxidant, and etc., thereby obtaining a core mixture; particles of the core mixture are coated with an inner coating layer comprising a PCM having a melting point below 60° C., forming a stable film or matrix which embeds the probiotic, thereby obtaining particles coated with the first PCM layer; the particles coated with the first PCM layer are coated with a second layer comprising at least one PCM whose the melting point is higher than that of the first PCM layer, wherein the second PCM layer may provide further resistance to higher temperature to the probiotics, thereby obtaining a particles coated with second PCM layer, the particles coated with second PCM layer are coated with more outer PCM layers, which the outer PCM layers have melting point higher than that of the second PCM layer conferring stability to the bacteria at higher temperatures on storage and shelf life at ambient temperature, wherein each outer PCM layer has melting point higher that its beneath PCM layer thereby obtaining layered particles coated with several PCM layers, the layered particles coated with several PCM layers are coated with an exterior coating layer comprising at least one polymer which is soluble in GI tract conferring stability to the bacteria on storage and shelf life at ambient temperature under the conditions of oxygen and humidity and/or protection against GI tract destructive parameters such as low pHs and enzymes, or further handling during production process such as coating a solid dosage form containing the layered particles wherein the at least one sugar may comprise, lactose, galactose or a mixture thereof, the at least one oligosaccharide or polysaccharides may comprise, galactan, maltodextrin, and trehalose, the stabilizer comprises L-cysteine base, the filler comprises lactose DC and/or microcrystalline cellulose, the binder comprises polyethylene glycol 1000 (PEG 1000), the first PCM coating layer may comprise PEG 1000, the second PCM coating layer may comprise polyethylene glycol 1500 (PEG 1500), the more outer PCM layers may comprise polyethylene glycol 2000 (PEG 2000), polyethylene glycol 4000 (PEG 4000) and polyethylene glycol 6000 (PEG 6000) respectively, the exterior coating layer may comprise carboxymethylcellulose (CMC) 7LFPH and/or carboxymethylcellulose (CMC) 7L2P. Both PCM layers as well as outermost layer may optionally further comprise at least one excipient, such as, for example, a plasticizer, a glidant including but not limited to silicon dioxide, lubricant and anti-adherents, including but not limited to microcrystalline cellulose, talc or titanium dioxide.

Another preferred process of manufacturing layered microencapsulated probiotic bacteria includes the following steps:

1. Drying mix of probiotics mixture, with at least one sugar and at least one oligosaccharide, and optionally other food grade additives such as stabilizers, fillers, antioxidant, and etc., thereby obtaining a core mixture.

2. Granulating the core mixture using melt of a binder, under either air or nitrogen environment thereby obtaining a core granule.

3. Coating particles of the core granule with an inner coating layer comprising a PCM thereby obtaining core granules coated with first PCM layer.

4. Coating the core granules coated with first PCM layer with a second PCM layer for thereby obtaining core granules coated with second PCM layer.

5. Coating the core granules coated with second PCM layer with additional more outer PCM layers thereby obtaining layered bacteria granules or microencapsules.

6. Coating the with an exterior coating layer which is soluble in the GI tract thereby obtaining layered particles containing probiotics showing superior stability against high temperature and oxygen and/or humidity on storage duration and during the shelf life and further handling during production process such as coating a solid dosage form containing the layered particles thus showing higher viability and vitality.

A mixture that comprises probiotic material is prepared and/or then converted to granules, e.g., by fluidized bed technology such as Glatt or turbo jet, Glatt or an Innojet coater/granulator, or a Huttlin coater/granulator, or a Granulex. The resulting granules, are microencapsulated by a first layer, which is a PCM then by a second layer with a PCM having a melting point higher than the first layer then coating with other PCMs where each layer has a melting point higher that the former layer and then finally coating with an outermost layer providing further protection against humidity and oxygen. Then resulting layered microencapsulated probiotics according to the above steps is introduced to a food product which may also undergo a heating step during its preparation process. Alternatively the above resulting microencapsulated probiotics can be added to a pharmaceutical or nutraceutical or nutritional dosage form such as particles, beads, microspheres, granules, mini-tablets, tablets, caplets, capsules, MUPS, syrups, beverages and alike which may be exposed to an ambient temperature during its preparation process such as coating process or packaging. During the exposure of the above resulted microencapsulated probiotics to ambient temperature, during the preparation process of the food product or pharmaceutical or nutraceutical or nutritional dosage form, the PCM layers, which are composed of different PCM varying in their melting points, form protecting layers surrounding the probiotics core granule preventing the transmission of the heat to the probiotics. Furthermore, after placing the food product or pharmaceutical or nutraceutical or nutritional product dosage forms containing the encapsulated particular probiotics prepared as described above on storage or shelf at ambient temperature, the probiotics show higher survival and viability during the storage thus providing longer shelf life. The invention thus provides a food product such as creams, biscuits creams, biscuit fill-in, chocolates, sauces, mayonnaise, dairy products and alike or pharmaceutical or nutraceutical or nutritional product dosage forms such as particles, beads, microspheres, granules, mini-tablets, tablets, caplets, capsules, MUPS, syrups, beverages and alike containing probiotics which survive the heating step needed during the preparation of the product for human uses. The product further will have a higher vitality and viability of probiotics and thus show a prolonged shelf life. The food product or pharmaceutical or nutraceutical or nutritional product dosage forms consist of: a) encapsulated granules, made of a mixture that comprises probiotic material which is dried and converted to core granules to be microencapsulated by a first layer, which is a PCM then by a second layer with a PCM having a melting point higher than the first layer then coating with other PCMs where each layer has a melting point higher that the former layer and then finally coating with an outermost layer providing further protection against humidity and oxygen and b) a food product or pharmaceutical or nutraceutical or nutritional product dosage forms to which the microencapsulated granules according to the present invention are previously added. Such a food product may contain high viability and vitality of probiotics even after long duration of storage at ambient temperature and thus may show a prolonged shelf life.

According to some demonstrative embodiments, there is provided a process for preparing probiotic bacteria capable of heating during manufacturing below 60° C. or preparing food or pharmaceutical or nutraceutical or nutritional product dosage forms with high rates of survivability. According to one embodiment of the present invention, the first step in making the probiotic food or pharmaceutical or nutraceutical or nutritional product dosage forms is preparing a core or granules comprising dried probiotic bacteria. These granules are then microencapsulated by different PCM layers. The first layer comprises at least one PCM having the lowest melting point. The second layer is then created comprising at least one PCM having a melting point higher than that of the first layer. The third layer is then created comprising one PCM having a melting point higher than that of the second layer. Additional PCM layer may be further subsequently created where each layer has a melting point which is higher than that of the former layer. The encapsulated granular/particular probiotics are then added to a food product or pharmaceutical or nutraceutical or nutritional product dosage forms before the final preparation. The food product or pharmaceutical or nutraceutical or nutritional product dosage form containing the encapsulated granular/particular probiotics may contain high viability and vitality of probiotics even after further preparation processes in which a heating process may be involved and long duration of storage at ambient temperature and thus may show a prolonged shelf life.

Layering is an important matter since the temperature of surroundings may be variable and not necessarily constant. Layered microencapsulation can make sure that the core will be substantially protected where it is exposed to varying thermal conditions where each layer having its own specific melting point may provide the core with maximum protection at each surrounding temperature.

In order to hinder the harmful effect of heat and thus the temperature increase of the product contacting the sensitive probiotic bacteria according to the present invention a layered microencapsulation technology using heat absorbing polymer has been used.

Generally a heat absorbing material (HAM) can be a kind of phase change material (PCM) having the ability to absorb energy in heat form at a specific temperature when its state changes. The absorption of heat is carried out upon melting process of PCM since the melting process is thermodynamically an endothermic process during which energy is absorbed by the material from surrounding causing cooling effect.

This heat can also be captured by energy storage material. HAM is a good energy storage material, which absorbs such excess heat. This excess of heat melts the HAM.

This character of the HAP does not allow the temperature of the product to increase until the HAP melts completely. Thus for a particular period of time (until the PCM melts completely) the temperature can be totally maintained.

In general, there are three modes of thermal energy storage by materials. These are sensible heat storage (SHS), latent heat storage (LHS) and bond energy storage (BES). SHS refers to the energy systems that store thermal energy without phase change. SHS occurs by adding heat to energy material and increasing its temperature. Heat is added from a heat source to the liquid or solid storage material. Heating of a material that undergoes a phase change (PCM), usually melting, is called the LHS. The amount of energy absorbed in the HLS depends upon the mass and latent heat of the material. In the LHS, the absorption operates isothermally at the phase change of the material.

Sensible Heat Storage

Every material stores energy within it as it is heated, and in this way is a “sensible heat storage material.” The energy stored can be quantified in terms of the heat capacity C, the temperature change
ΔT=final temperature−initial temperature, and the amount of additional heat stored ΔQ, according to the second law of thermodynamics as follows;

$$\begin{matrix}Δ Q = V ρ {C}_{p} Δ T \\ = {mc}_{p} Δ T\end{matrix} \qquad ( 1 )$$

where

ΔQ=sensible heat stored in the material (J, Btu)

V=volume of substance (m³, ft³)

p=density of substance (kg/m³, lb/ft³)

m=mass of substance (kg, lb)

Cₚ=specific heat capacity of the substance (J/kg° C., Btu/lb° F.)

ΔT=temperature change (° C., ° F.)

Clearly, other factors being equal, the higher the heat capacity (C) of a material, the greater will be the energy stored (ΔQ) for a given temperature rise (ΔT).

Phase-Change Materials (PCM)

Phase change material is a latent heat storage material but can also store sensible heat. They use chemical bonds to absorb heat. The thermal energy transfer occurs when a material changes from a solid to a liquid or from a liquid to a solid. This is called a change in state, or “phase”. The various phase changes that can occur are melting, lattice change and etc.

Initially, these solid-liquid PCMs perform like conventional storage materials; their temperature rises as they absorb the heat from the surroundings. Unlike conventional (sensible) storage materials, when PCMs reach the temperature at which they change phase (their melting point) they absorb large amounts of heat without getting hotter.

PCMs absorb heat while maintaining a nearly constant temperature. They absorb 5 to 14 times more heat per unit volume than sensible storage materials. Thermal energy is generally absorbed as latent heat-by change of phase of medium. As a result temperature of the medium remains constant since it undergoes an endothermic phase transformation.

Each PCM has a melting temperature at which point it will transform from a solid to a liquid retaining the latent heat of fusion produced from the endothermic process. When the temperature is higher than this melting point, the material will liquefy absorbing the thermal energy from the surrounding environment at a constant rate.

Every material is actually a Phase Change Material (PCM) because at certain combinations of pressure and temperature every material can change its aggregate state (solid, liquid, gaseous). In a change of aggregate state, a large amount of energy, the so-called latent heat, can be absorbed at an almost constant temperature.

Although all materials increase their heat content Q as the temperature is increased, a very large increase in Q occurs when materials change phase. For example, the heat content of water increases considerably as it goes through the phase change from ice to liquid; this is the familiar melting process. The step in Q at the phase change is the latent heat associated with the transition, usually represented as Δtrs H. The step in Q at the transition is in addition to the sensible heat storage capacity of the material.

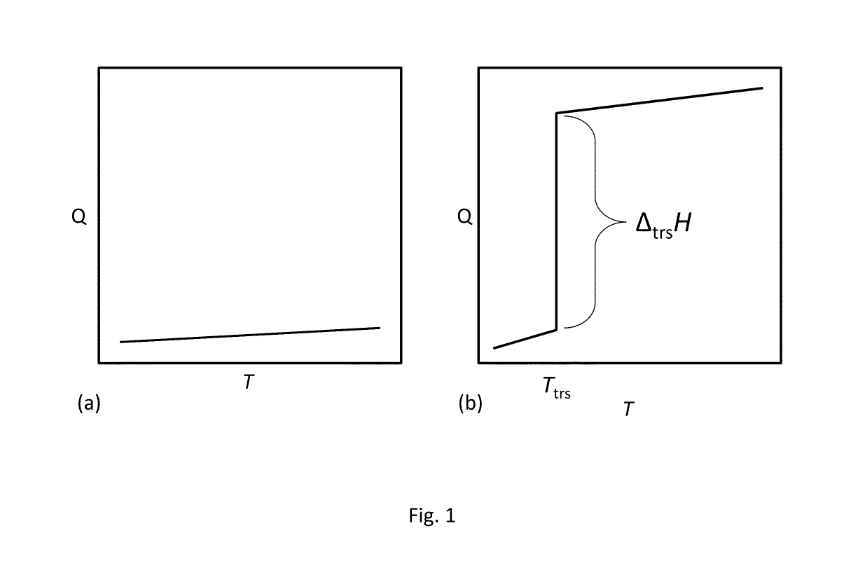

FIG. 1 shows heat content Q as a function of temperature T. (a) Q increases with increasing temperature, even if there is no phase transition, as in a sensible heat storage material. (b) When the material undergoes a phase transition at temperature Ttrs, a dramatic increase in Q occurs; its jump corresponds to the value of the latent heat of the transition ΔtrsH as indicated on the diagram. This large increase in Q can be used to advantage in phase-change materials for heat storage.

A phase change can lead to a much larger quantity of energy absorption, compared with sensible storage alone. The comparison for water is quite useful. Pure water has a heat capacity of 4.2 J K-1 g-1, so for a 1° C. temperature rise, 1 g of water can store 4.2 J. However, the latent heat associated with melting of ice is 330 J g-1. So taking 1 g of ice from just below its melting point to just above (with a total temperature difference of 1° C.) absorbs 334 J (latent heat plus 4.2 J from sensible heat storage), about 80 times as much as the sensible heat storage capacity alone.

Solid-solid PCMs absorb and release heat in the same manner as solid-liquid PCMs. These materials do not change into a liquid state under normal conditions. They merely soften or harden. Relatively few of the solid-solid PCMs that have been identified are suitable for thermal storage applications.

In order PCMs to be useful for layering in the structure of microencapsules according to the present invention, PCM candidates must be able to fulfill a number of desirable criteria; and possess suitable properties for their application.

First it is important that the phase transition temperatures of the PCM (i.e. for cooling) are in the required temperature range suitable for its application. They must have their phase transition in the temperature range at which the sensitive active materials will be exposed. This range of temperatures determines the range of temperatures in which the protection should take place. According to the present invention the melting point of PCM should be below 90° C., preferably below 80° C., more preferably below 70° C. and most preferably below 60° C.

For example, at ordinary pressures water if a heat storage system were required to provide protection (cooling effect) in the temperature range of 40-60° C. a PCM which has a melting point at 80° C. could operate only as a sensible heat storage material, not as a phase-change material. Depending on the choice of material the operating temperature range for PCM can be sufficiently large.

Another important characteristic of PCM which can be useful in the present invention is the latent heat of fusion of the material. The melting process must produce a high latent heat of fusion per unit volume. The higher the latent heat of fusion the higher will be the amount of energy absorbed by PCM during the phase change process (melting process). The amount of energy absorbed (E) by a PCM in this case depends upon mass (m) and latent heat of fusion of the material (ΔH). Thus,

E=mΔH

The absorption operates isothermally at the melting point of the material. If isothermal operation at the phase change temperature is difficult, the system operates over range of temperatures T1 to T2 that includes the melting point. The sensible heat contributions have to be considered and the amount of energy absorbed during the phase change is given by;

$$E = m [ \{ {\int }_{T 1}^{T +} Cps d T \} + Δ H + \{ {\int }_{Tm}^{T 2} Cpl d T \} ]$$

Where Cps and Cpl represents the specific heat capacities of the solid and liquid phases and Tm is the melting point. In addition to the latent heat absorbed, significant sensible heat produced from the phase change must also be absorbed. The reasons why PCM is a suggested material in the present invention is the fact that thermal storage capacity per unit mass and unit volume for small temperature differences is sufficiently high to provide heat sensitive active material with maximum protection against heating by its cooling affect.

It is also important to select a phase change material with a high rate of crystal growth so that during the coating process PCM can have high degree of crystallinity therefore a maximum latent heat of fusion may be obtained for maximum cooling effect. Method to enhance the crystallization of PCM during the coating process includes introducing nucleating agents as catalysts within the PCM mixture to help increase the rate of crystal growth.

The thermal properties of a PCM including melting point and latent heat of fusion can be comprehensively studied before selecting the most appropriate PCM for layering and microencapsulation. The methods most commonly used to assess the thermal characteristics of a PCM are Differential Thermal Analysis (DTA) and Differential Scanning calorimetry (DSC). Both of these techniques involve measuring the latent heat of fusion and melting temperature characteristics of PCMs. The analysis uses a recommended reference material, Al2O3, and a PCM sample, which are both heated at a constant rate. The temperature difference recorded between the two materials is proportional to the rate of heat flow in either material. The result is presented on a DSC graph, where the latent heat of fusion is calculated from the area under the curve; and the melting temperature is estimated from the gradient at the steepest point on the curve.

Another important characteristic of a PCM according to the present invention is the length of time during which energy can be kept absorbed. The longer the time to complete fusion the higher will be the efficiency of the PCM in absorption process. This length of time is determined by the thickness of the coating layer, the amount of latent heat of fusion per unit weight as well as specific heat capacity of PCM. Another important characteristic of a PCM is its volumetric energy capacity, or the amount of energy absorbed per unit volume. The smaller the volume, the better is the absorption system. Therefore, a good PCM should have a high heat of fusion per unit weight, a long absorption time and a small volume per unit of absorbed energy.

If mass specific heat capacity is not small, denser materials have smaller volumes and correspondingly an advantage of larger energy capacity per unit volume.

Other considerations include the suitability and compatibility of materials used for food, pharmaceutical and nutraceutical applications. The substance must be compatible with the surrounding materials being used in the formulation of inner core.

PCMs for Layering Process

There are a wide range of polymeric and non-polymeric organic materials which can be applied as appropriate PCM in the microencapsulation composition according to the present invention. Different PCMs having either different or same chemical structure but varying in their melting points are used in the layering and microencapsulation process. By this way a wide range of temperatures is covered within which the cooling effect can be provided.

The most suitable materials which can act as an appropriate PCM according to the present invention are alkenes, waxes, esters, fatty acids, alcohols, and glycols, each with varying performance and properties independent of each other.

Example of materials that may be used as phase change material is selected from the group consisting of alkenes such as paraffin wax which is composed of a chain of alkenes, normal paraffins of type CₙH₂ₙ₊₂ which are a family of saturated hydrocarbons which are waxy solids having melting point in the range of 23-67° C. (depending on the number of alkanes in the chain); both natural waxes (which are typically esters of fatty acids and long chain alcohols) and synthetic waxes (which are long-chain hydrocarbons lacking functional groups) such as bee wax, carnauba wax, japan wax, bone wax, paraffin wax, chinese wax, lanolin (wool wax), shellac wax, spermaceti, bayberry wax, candelilla wax, castor wax, esparto wax, jojoba oil, ouricury wax, rice bran wax, soy wax, ceresin waxes, montan wax, ozocerite, peat waxes, microcrystalline wax, petroleum jelly, polyethylene waxes, fischer-tropsch waxes, chemically modified waxes, substituted amide waxes; polymerized α-olefins; hydrogenated vegetable oil, hydrogenated castor oil; fatty acids, such as lauric acid, myristic acid, palmitic acid, palmitate, palmitoleate, hydroxypalmitate, stearic acid, arachidic acid, oleic acid, stearic acid, sodium stearat, calcium stearate, magnesium stearate, hydroxyoctacosanyl hydroxystearate, oleate esters of long-chain, esters of fatty acids, fatty alcohols, esterified fatty diols, hydroxylated fatty acid, hydrogenated fatty acid (saturated or partially saturated fatty acids), aliphatic alcohols, phospholipids, lecithin, phosphathydil cholin, triesters of fatty acids for example triglycerides received from fatty acids and glycerol (1,2,3-trihydroxypropane) including fats and oils such as coconut oil, hydrogenated coconut oil, cacao butter (also called theobroma oil or theobroma cacao); eutectics such as fatty acid eutectics which are a mixture of two or more substances which both possess reliable melting and solidification behaviour; glycols such as polyethylene glycol, polyethylene oxides, Poloxamers which are block-co-polymers of polyethylene oxide and polypropylene glycol (Lutrol F), block-co-polymers of polyethylene glycol and polyesters, and a combination thereof.

Blend polymer can also be used as an appropriate PCM. The blend can be either miscible or immiscible where the former generally results only in one melting point whereas the latter may show separated melting points attributed to the pure polymers.

Intermediate Layers

According to some demonstrative embodiments of the invention, the layered microcapsules prepared according to the present invention may optionally and preferably be separated from each other by a polymer film layer which may be soluble in the GI tract. Example of materials that may be used for the outermost coating layer are selected from the group consisting of water soluble or erodible polymers such as, for example, Povidone (PVP: polyvinyl pyrrolidone), Copovidone (copolymer of vinyl pyrrolidone and vinyl acetate), polyvinyl alcohol, Kollicoat Protect (BASF) which is a mixture of Kollicoat IR (a polyvinyl alcohol (PVA)-polyethylene glycol (PEG) graft copolymer) and polyvinyl alcohol (PVA), Opadry AMB (Colorcon) which is a mixture based on PVA, Aquarius MG which is a cellulose-based polymer containing natural wax, lecithin, xanthan gum and talc, low molecular weight HPC (hydroxypropyl cellulose), low molecular weight HPMC (hydroxypropyl methylcellulose) such as hydroxypropylcellulose (HPMC E3 or E5) (Colorcon), methyl cellulose (MC), low molecular weight carboxy methyl cellulose (CMC), low molecular weight carboxy methyl ethyl cellulose (CMEC), low molecular weight hydroxyethylcellulose (HEC), low molecular weight hydroxyl ethyl methyl cellulose (HEMC), low molecular weight hydroxymethylcellulose (HMC), low molecular weight hydroxymethyl hydroxyethylcellulose (HMHEC), low viscosity of ethyl cellulose, low molecular weight methyl ethyl cellulose (MEC), gelatin, hydrolyzed gelatin, polyethylene oxide, water soluble gums, water soluble polysaccharides, acacia, dextrin, starch, modified cellulose, water soluble polyacrylates, polyacrylic acid, polyhydroxyethylmethacrylate (PHEMA) and polymethacrylates and their copolymers, pH-sensitive polymers for example enteric polymers including phthalate derivatives such as acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene-maleic acid dibutyl phthalate copolymer, styrene-maleic acid/polyvinylacetate phthalate copolymer, styrene and maleic acid copolymers, polyacrylic acid derivatives such as acrylic acid and acrylic ester copolymers, polymethacrylic acid and esters thereof, polyacrylic and methacrylic acid copolymers, shellac, and vinyl acetate and crotonic acid copolymers. Preferred pH-sensitive polymers include shellac, phthalate derivatives, CAT, HPMCAS, polyacrylic acid derivatives, particularly copolymers comprising acrylic acid and at least one acrylic acid ester, Eudragit S™ (poly(methacrylic acid, methyl methacrylate)1:2); Eudragit L™ which is an anionic polymer synthesized from methacrylic acid and methacrylic acid methyl ester, Eudragit L100™ (poly(methacrylic acid, methyl methacrylate)1:1); Eudragit L30D™, (poly(methacrylic acid, ethyl acrylate)1:1); and Eudragit L100-55™ (poly(methacrylic acid, ethyl acrylate)1:1), polymethyl methacrylate blended with acrylic acid and acrylic ester copolymers, alginic acid and alginates such as ammonia alginate, sodium, potassium, magnesium or calcium alginate, vinyl acetate copolymers, polyvinyl acetate 30D (30% dispersion in water), a poly(dimethylaminoethylacrylate) which is a neutral methacrylic ester available from Rohm Pharma (Degusa) under the name “Eudragit E™, and/or any combination thereof.

Exterior Coating Layer

According to further features in any of the embodiments of the invention, the layered microcapsules prepared according to the present invention may optionally and preferably further comprises an outermost (exterior) coating layer which is preferably soluble in the GI tract. The exterior coating layer may provide further with additional protection against penetration of either humidity or oxygen or both into the core during both production process as well as shelf life of the final product.

Example of materials that may be used for the outermost coating layer is selected from the group consisting of water soluble or erodible polymers such as, for example, Povidone (PVP: polyvinyl pyrrolidone), Copovidone (copolymer of vinyl pyrrolidone and vinyl acetate), polyvinyl alcohol, Kollicoat Protect (BASF) which is a mixture of Kollicoat IR (a polyvinyl alcohol (PVA)-polyethylene glycol (PEG) graft copolymer) and polyvinyl alcohol (PVA), Opadry AMB (Colorcon) which is a mixture based on PVA, Aquarius MG which is a cellulose-based polymer containing natural wax, lecithin, xanthan gum and talc, low molecular weight HPC (hydroxypropyl cellulose), low molecular weight HPMC (hydroxypropyl methylcellulose) such as hydroxypropylcellulose (HPMC E3 or E5) (Colorcon), methyl cellulose (MC), low molecular weight carboxy methyl cellulose (CMC), low molecular weight carboxy methyl ethyl cellulose (CMEC), low molecular weight hydroxyethylcellulose (HEC), low molecular weight hydroxyl ethyl methyl cellulose (HEMC), low molecular weight hydroxymethylcellulose (HMC), low molecular weight hydroxymethyl hydroxyethylcellulose (HMHEC), low viscosity of ethyl cellulose, low molecular weight methyl ethyl cellulose (MEC), gelatin, hydrolyzed gelatin, polyethylene oxide, water soluble gums, water soluble polysaccharides, acacia, dextrin, starch, modified cellulose, water soluble polyacrylates, polyacrylic acid, polyhydroxyethylmethacrylate (PHEMA) and polymethacrylates and their copolymers,

pH-sensitive polymers for example enteric polymers including phthalate derivatives such as acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene-maleic acid dibutyl phthalate copolymer, styrene-maleic acid/polyvinylacetate phthalate copolymer, styrene and maleic acid copolymers, polyacrylic acid derivatives such as acrylic acid and acrylic ester copolymers, polymethacrylic acid and esters thereof, polyacrylic and methacrylic acid copolymers, shellac, and vinyl acetate and crotonic acid copolymers. Preferred pH-sensitive polymers include shellac, phthalate derivatives, CAT, HPMCAS, polyacrylic acid derivatives, particularly copolymers comprising acrylic acid and at least one acrylic acid ester, Eudragit S™ (poly(methacrylic acid, methyl methacrylate)1:2); Eudragit L™ which is an anionic polymer synthesized from methacrylic acid and methacrylic acid methyl ester, Eudragit L100™ (poly(methacrylic acid, methyl methacrylate) 1:1); Eudragit L30D™, (poly(methacrylic acid, ethyl acrylate)1:1); and Eudragit L100-55™ (poly(methacrylic acid, ethyl acrylate)1:1), polymethyl methacrylate blended with acrylic acid and acrylic ester copolymers, alginic acid and alginates such as ammonia alginate, sodium, potassium, magnesium or calcium alginate, vinyl acetate copolymers, polyvinyl acetate 30D (30% dispersion in water), a poly(dimethylaminoethylacrylate) which is a neutral methacrylic ester available from Rohm Pharma (Degusa) under the name “Eudragit E™, and/or a mixtures thereof.

Substrate:

According to a preferred embodiment of the invention, the heat sensitive active material (including probiotic bacteria) in the granule core are mixed with a substrate. The substrate preferably comprises at least one material that may be also a supplement agent and/or a stabilizer for the probiotic bacteria. The substrate may comprise monosaccharides such as trioses including ketotriose (dihydroxyacetone) and aldotriose (glyceraldehyde), tetroses such as ketotetrose (erythrulose), aldotetroses (erythrose, threose) and ketopentose (ribulose, xylulose), pentoses such as aldopentose (ribose, arabinose, xylose, lyxose), deoxy sugar (deoxyribose) and ketohexose (psicose, fructose, sorbose, tagatose), hexoses such as aldohexose (allose, altrose, glucose, mannose, gulose, idose, galactose, talose), deoxy sugar (fucose, fuculose, rhamnose) and heptose such as (sedoheptulose), and octose and nonose (neuraminic acid). The substrate may comprise multiple saccharides such as 1) disaccharides, such as sucrose, lactose, maltose, trehalose, turanose, and cellobiose, 2) trisaccharides such as raffinose, melezitose and maltotriose, 3) tetrasaccharides such as acarbose and stachyose, 4) other oligosaccharides such as fructooligosaccharide (FOS), galactooligosaccharides (GOS) and mannan-oligosaccharides (MOS), 5) polysaccharides such as glucose-based polysaccharides/glucan including glycogen starch (amylose, amylopectin), cellulose, dextrin, dextran, beta-glucan (zymosan, lentinan, sizofiran), and maltodextrin, fructose-based polysaccharides/fructan including inulin, levan beta 2-6, mannose-based polysaccharides (mannan), galactose-based polysaccharides (galactan), and N-acetylglucosamine-based polysaccharides including chitin. Other polysaccharides may be comprised, including gums such as arabic gum (gum acacia).

According to preferred embodiments of the present invention, the core further comprises an antioxidant. Preferably, the antioxidant is selected from the group consisting of cysteine hydrochloride, cystein base, 4,4-(2,3 dimethyl tetramethylene dipyrocatechol), tocopherol-rich extract (natural vitamin E), α-tocopherol (synthetic Vitamin E), β-tocopherol, γ-tocopherol, δ-tocopherol, butylhydroxinon, butyl hydroxyanisole (BHA), butyl hydroxytoluene (BHT), propyl gallate, octyl gallate, dodecyl gallate, tertiary butylhydroquinone (TBHQ), fumaric acid, malic acid, ascorbic acid (Vitamin C), sodium ascorbate, calcium ascorbate, potassium ascorbate, ascorbyl palmitate, and ascorbyl stearate. Comprised in the core may be citric acid, sodium lactate, potassium lactate, calcium lactate, magnesium lactate, anoxomer, erythorbic acid, sodium erythorbate, erythorbin acid, sodium erythorbin, ethoxyquin, glycine, gum guaiac, sodium citrates (monosodium citrate, disodium citrate, trisodium citrate), potassium citrates (monopotassium citrate, tripotassium citrate), lecithin, polyphosphate, tartaric acid, sodium tartrates (monosodium tartrate, disodium tartrate), potassium tartrates (monopotassium tartrate, dipotassium tartrate), sodium potassium tartrate, phosphoric acid, sodium phosphates (monosodium phosphate, disodium phosphate, trisodium phosphate), potassium phosphates (monopotassium phosphate, dipotassium phosphate, tripotassium phosphate), calcium disodium ethylene diamine tetra-acetate (calcium disodium EDTA), lactic acid, trihydroxy butyrophenone and thiodipropionic acid and mixtures thereof. According to one preferred embodiment, the antioxidant is cystein base.

According to some embodiments of the present invention, the core further comprises a filler and binder. Examples of fillers include, for example, microcrystalline cellulose, a sugar, such as lactose, glucose, galactose, fructose, or sucrose; dicalcium phosphate; sugar alcohols such as sorbitol, manitol, mantitol, lactitol, xylitol, isomalt, erythritol, and hydrogenated starch hydrolysates; corn starch; potato starch; sodium carboxymethycellulose, ethylcellulose and cellulose acetate, or a mixture thereof. More preferably, the filler is lactose.

Examples of binders include Povidone (PVP: polyvinyl pyrrolidone), Copovidone (copolymer of vinyl pyrrolidone and vinyl acetate), polyvinyl alcohol, low molecular weight HPC (hydroxypropyl cellulose), low molecular weight HPMC (hydroxypropyl methylcellulose), low molecular weight carboxy methyl cellulose, low molecular weight hydroxyethylcellulose, low molecular weight hydroxymethylcellulose, gelatin, hydrolyzed gelatin, polyethylene oxide, acacia, dextrin, starch, and water soluble polyacrylates and polymethacrylates, low molecular weight ethylcellulose, fatty acids, waxes, hydrogenated oils, polyethylene glycol, block-copolymer of polyethylene glycol and polypropylene glycol (Poloxamer) or a mixture thereof.

According to preferred embodiments of the present invention PCM layers as well as outermost layer may optionally further comprise at least one excipient, such as, for example, a plasticizer, a glidant including but not limited to silicon dioxide, lubricant and anti-adherents, including but not limited to microcrystalline cellulose, talc or titanium dioxide or combinations thereof.

According to preferred embodiments of the present invention the dosage form containing stabilized probiotic granules or miroencapsules is further optionally and preferably coated by an enteric polymer which may further provide protection through GI tract destructive parameters such as low pHs and enzymes.

Example of materials that may be used for coating the dosage form is selected from the group consisting of pH-sensitive polymers for example, acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS), methylcellulose phthalate (MCP), polyvinyl acetate phthalate (PVAcP), polyvinyl acetate hydrogen phthalate, sodium CAP, starch acid phthalate, cellulose acetate trimellitate (CAT), styrene and maleic acid copolymers, styrene-maleic acid dibutyl phthalate copolymer, styrene-maleic acid/polyvinylacetate phthalate copolymer, polyacrylic acid derivatives such as acrylic acid and acrylic ester copolymers, polymethacrylic acid and esters thereof, polyacrylic and methacrylic acid copolymers, polyacrylic acid derivatives such as particularly copolymers comprising acrylic acid and at least one acrylic acid ester, Eudragit S™ (poly(methacrylic acid, methyl methacrylate)1:2); Eudragit L™ which is an anionic polymer synthesized from methacrylic acid and methacrylic acid methyl ester), Eudragit L100 acid, methyl methacrylate)1:1); Eudragit L30D™, (poly(methacrylic acid, ethyl acrylate)1:1); and Eudragit L100-55 acid, ethyl acrylate)1:1), polymethyl methacrylate blended with acrylic acid and acrylic ester copolymers, alginic acid and alginates such as ammonia alginate, sodium, potassium, magnesium or calcium alginate.

### Example 1

First the effect of melting and re-crystallization of PEG with different molecular weight was investigated. For this purpose different molecular weights of PEG were melted and cooled for re-crystallizing followed by melting again. The cooling took place by both slow and fast rate. The effect of cooling rate was determined by differential scanning calorimetry method (DSC).

For slow cooling, the polymer melt was left at room temperature to slowly be re-crystallized and left at freezer for fast cooling.

DSC was carried out by heating rate of 10° C./min in a temperature range of 10-100° C. A specimen of 5-10 mg was use for DSC tests. An empty aluminum pan was used as the control for DSC analysis.

The results of the effect of cooling rate on melting point of PEG with different molecular weight are summarized in the following table and shown in following thermograms.

| | | Melting point | Melting point |
|---|---|---|---|
| | Initial Melting | after fast cooling | after slow |
| Molecular | point | rate | cooling rate |
| weight of PEG | (T_{M}) [° C.] | (T_{M}) [° C.] | (T_{M}) [° C.] |
| PEG 1000 | 40.7 | 32.3 | 41.4 |
| PEG 1500 | 52.2 | 45.7 | 52.9 |
| PEG 2000 | 57.8 | 57.7 | 57.7 |
| PEG 4000 | 60.3 | 65.8 | 63.9 |
| PEG 6000 | 65.9 | 66.8 | 64.4 |
| PEG 8000 | 67.7 | 68.1 | 67.4 |

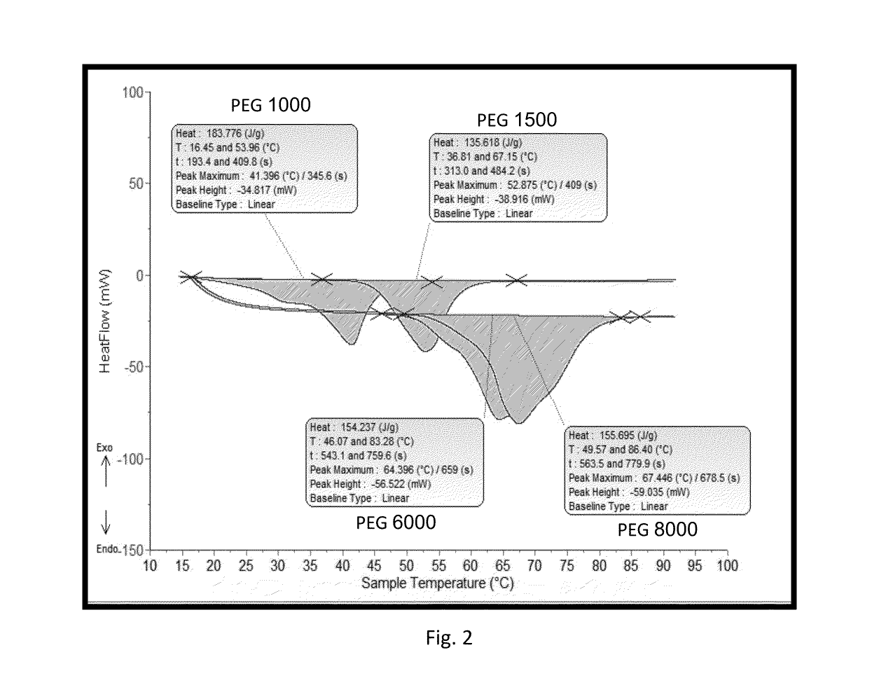

FIG. 2 illustrates the effect of slow cooling rate on melting point of PEG with different molecular weights, including PEG 1000, PEG 1500, PEG 6000 and PEG 8000.

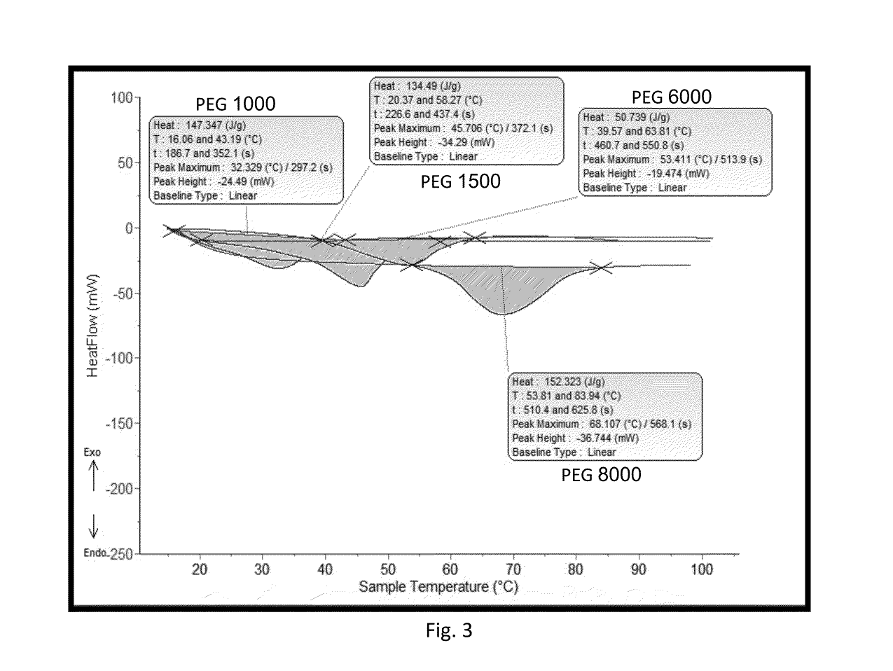

FIG. 3 illustrates the effect of fast cooling rate on melting point of PEG with different molecular weights, including PEG 1000, PEG 1500, PEG 6000 and PEG 8000.

### Example 2

In order to determine the relationship between the layers and especially the nature of the interfacial relationship between the layers, a laminated film structure was prepared using different molecular weights of PEG (polyethylene glycol). This laminated structure (laminar substance) was compared with blends prepared using PEG with the same molecular weights. This was doe by thermal characterization of laminated structure as compared to blend compositions using a differential scanning calorimetry method (DSC).

For the preparation of different blends the polymers first melted and mixed and then the was allowed to be re-crystallized at different cooling rate. For slow cooling, the resulting mixture was left at room temperature to slowly be re-crystallized and left at freezer for fast cooling.

The results of the effect of cooling rate on melting point of each PEG (with different molecular weight) in the blend are shown in following thermograms.

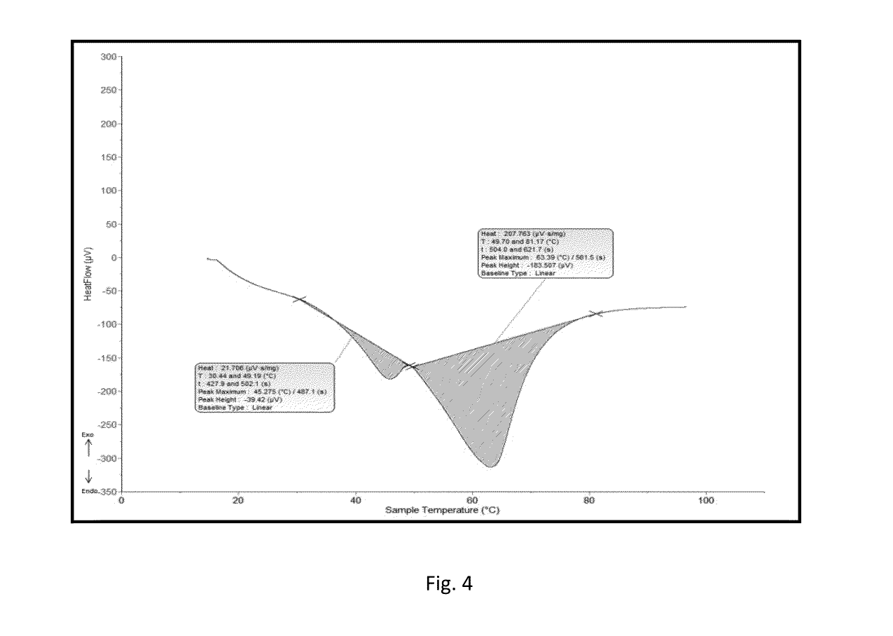

FIG. 4 illustrates the effect of slow cooling on melting point of a blend comprising PEG 1500 and PEG 6000

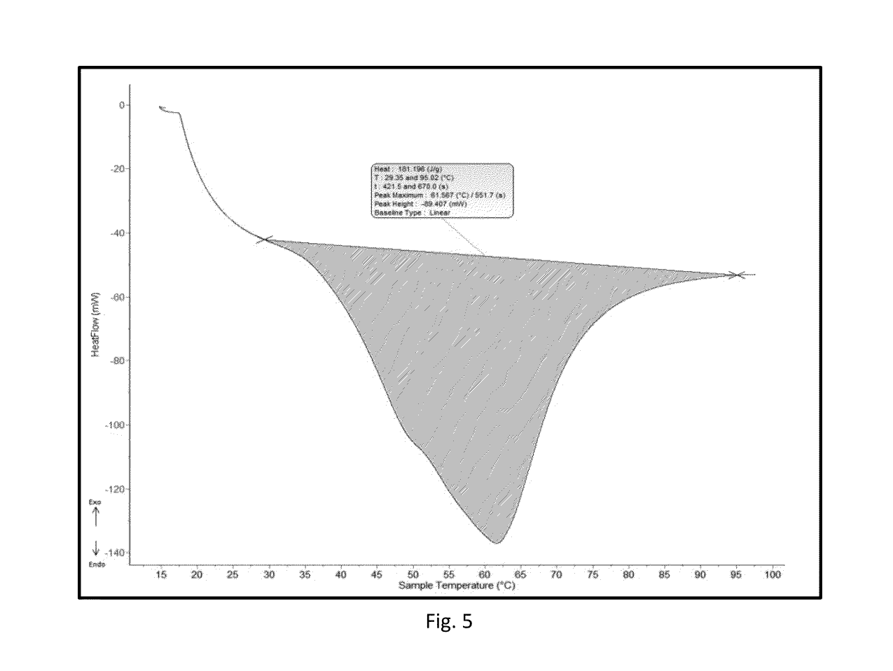

FIG. 5 illustrates the effect of fast cooling on melting point of a blend comprising PEG 1500 and PEG 6000

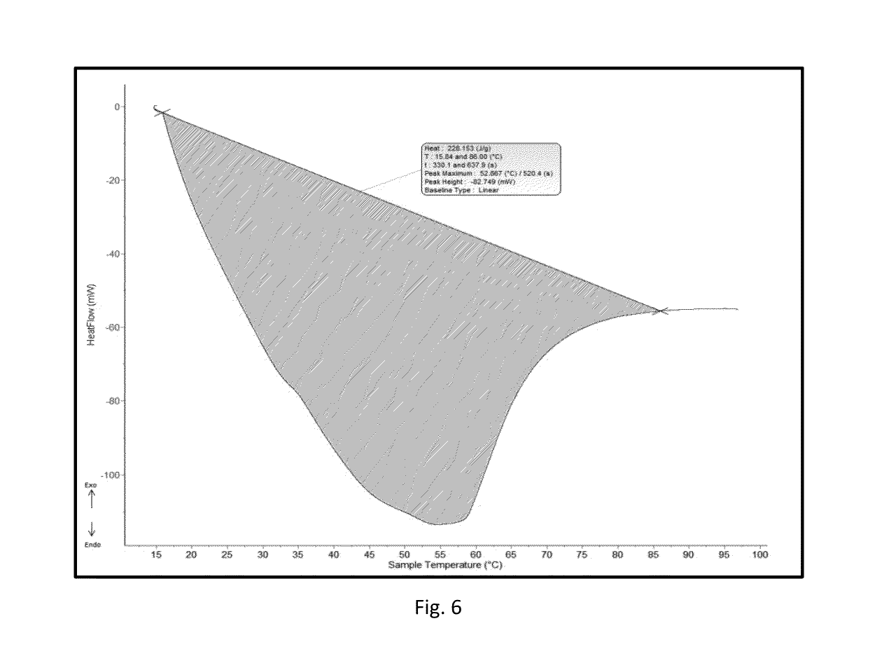

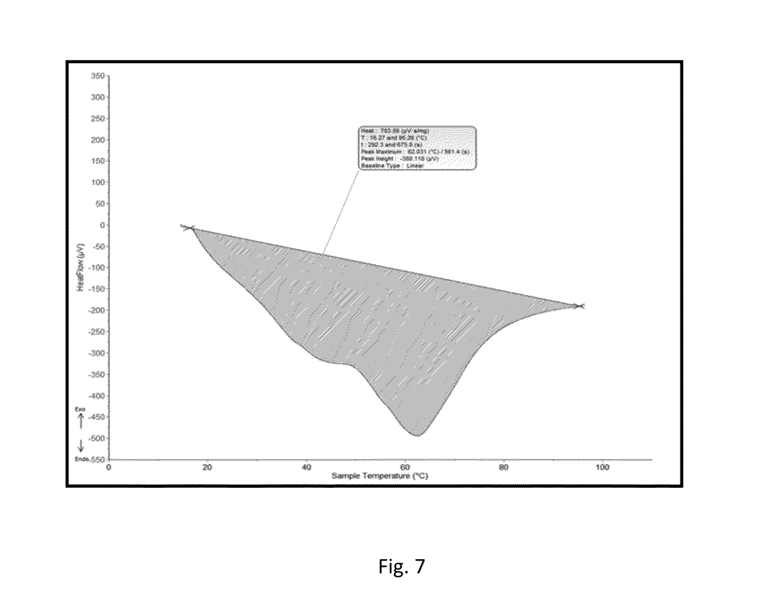

FIG. 6 illustrates the effect of fast cooling on melting point of a blend comprising PEG 1000 and PEG 6000

FIG. 7 illustrates the effect of fast cooling on melting point of a blend comprising PEG 1000 and PEG 2000

### Example 3

For the preparation of different laminated structures the polymers are first melted and poured, layer onto layer, in an order of increasing molecular weight of PEG where each layer was allowed to be properly re-crystallized (in the freezer) before pouring the next layer.

The melting points of each PEG (with different molecular weights) was then determined by testing the resulting laminated structure using DSC method. The DSC thermograms of different laminated structure are shown as follows;

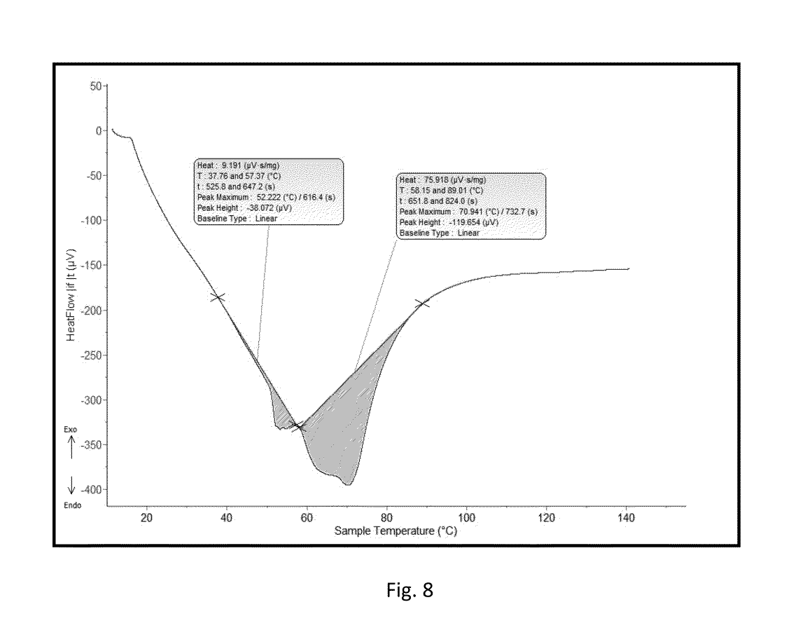

FIG. 8 illustrates a thermogram of a laminated structure comprising PEG 1000 and PEG 2000

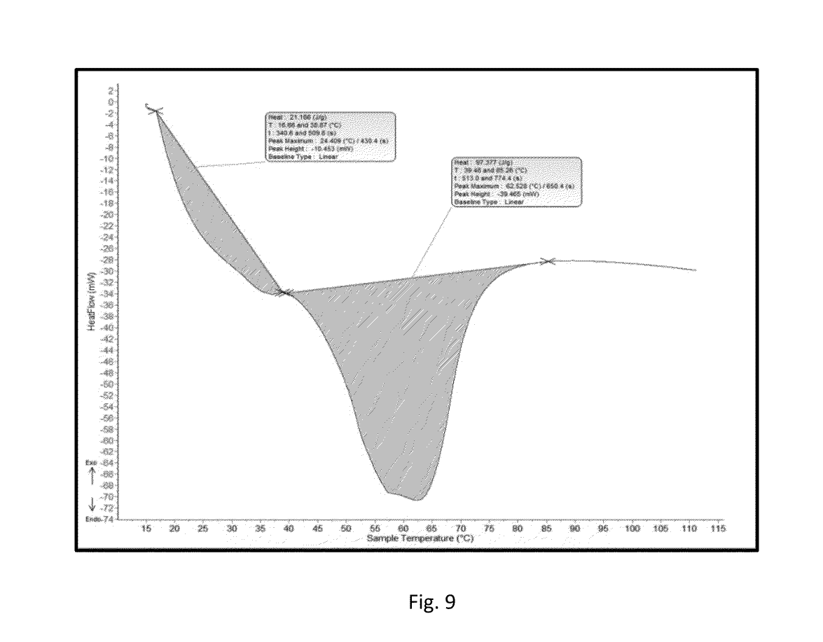

FIG. 9 illustrates a thermogram of a laminated structure comprising PEG 1000, PEG 2000 and PEG 4000

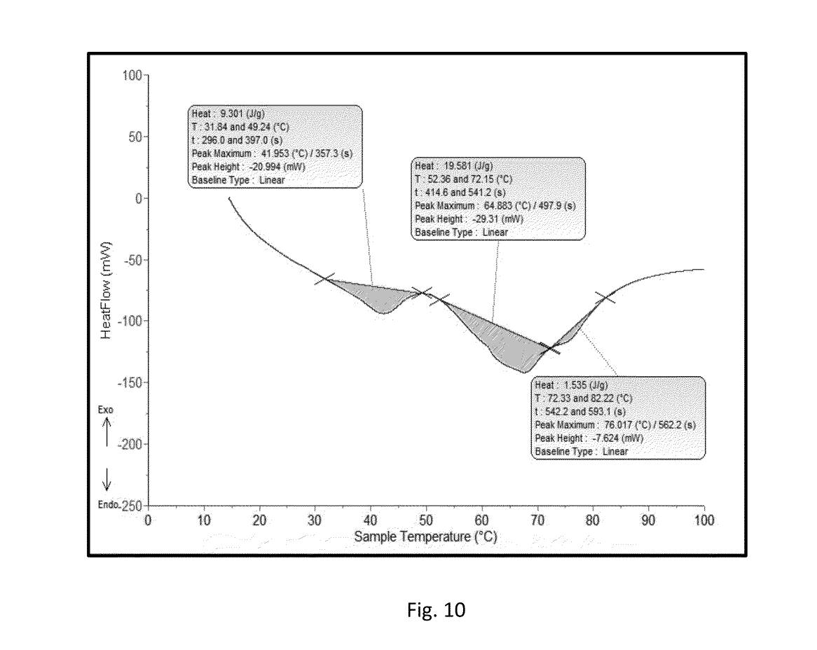

FIG. 10 illustrates a thermogram of a laminated structure comprising PEG 1000, PEG 2000 and PEG 8000

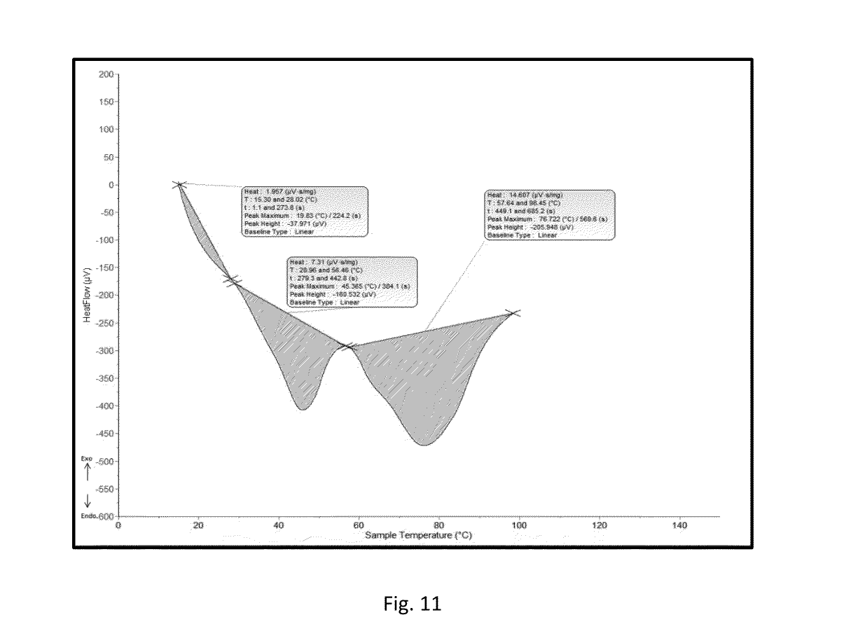

FIG. 11 illustrates a thermogram of a laminated structure comprising PEG 1000, PEG 4000 and PEG 8000

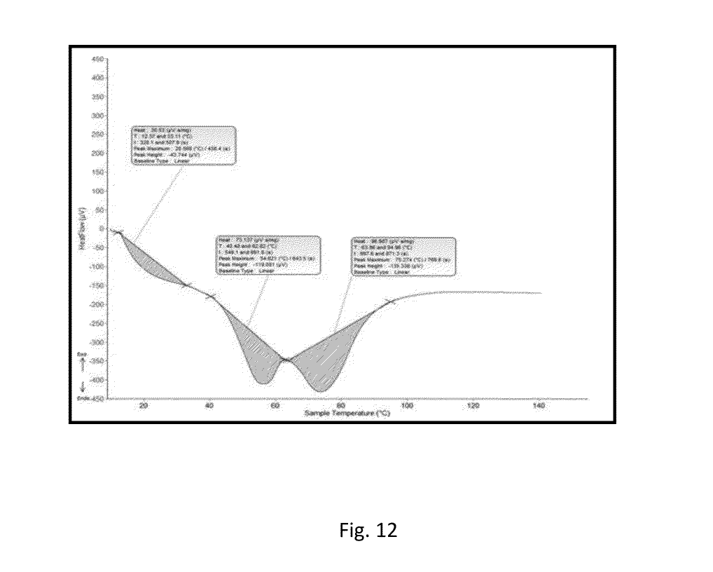

FIG. 12 illustrates a thermogram of a laminated structure comprising PEG 1500, PEG 6000 and PEG 8000

### Example 4

TABLE 1
the list of materials used in microencapsulation process of the probiotic
according to the present invention in this non-limiting Example

| Materials: | |
|---|---|
| L. Gasseri | Probiotic Bacteria |
| Maltodextrin | Substrate |
| Trehalose | Substrate |
| Cystein-HCl | Stabilizer- Antioxidant |
| Microcrystalline cellulose (MCC) | Glidant |
| Polyethylene glycol 1000 | Binder |
| Polyethylene glycol | PCM |

Polyethylene Glycol with different molecular weights was used as PCM for stratifying probiotic core granules. The molecular weight and the melting points of this series of PEG used in this experiment have been summarized in Table 2.

First a mixture of trehalose (80 g), probiotic bacteria L. gasseri 57C (Biomed) (60 g), cystein-HCl (3 g) and maltodextrin (157 g) was loaded into Innojet Ventilus (Innojet IEV2.5 V2). Then PEG 1000 (135 g) was melted at 50° C. and microcrystalline cellulose (MCC PH 105) (13.5 g) was added into the melted PEG and homogenized to obtain a uniform dispersion. Then the resulting homogenous dispersion was sprayed onto the above dry mixture under an inert atmosphere using nitrogen. The temperatures of pump head, liquid, and spray pressure were set at room temperature. By theses means granulates were obtained based on a melt granulation. The resulting granules were then coated by homogenous dispersion of PEG 1000 melt (43.5 g) and MCC PH 105 (4.4 g) under an inert atmosphere using nitrogen to obtain granules coated by the first PCM coating layer. PEG 1500 (47.9 g) was melted and then MCC PH 105 (4.8 g) was added and homogenized to obtain a homogenous dispersion. The latter homogeneous dispersion was then sprayed onto the above granules coated by the first PCM coating layer to obtain granules coated by the second PCM coating layer. PEG 2000 (52.6 g) was melted and then MCC PH 105 (5.3 g) was added and homogenized to obtain a homogenous dispersion. The latter homogeneous dispersion was then sprayed onto the above granules coated by the second PCM coating layer to obtain granules coated by the third PCM coating layer. The resulting granules coated by the third PCM coating layer was discharged and kept in freezer for 2 hours. Then frozen granules coated by the third PCM coating layer were loaded again into Innojet Ventilus (Innojet IEV2.5 V2). PEG 4000 (40.9 g) was melted and MCC PH 105 (4.1 g) was added and homogenized to obtain a homogenous dispersion. The latter homogeneous dispersion was then sprayed onto the above granules coated by the third PCM coating layer to obtain granules coated by the fourth PCM coating layer. PEG 6000 (56 g) was melted and MCC PH 105 (5.6 g) was added and homogenized to obtain a homogenous dispersion. The latter homogeneous dispersion was then sprayed onto the above granules coated by the fourth PCM coating layer to obtain granules coated by the fifth PCM coating layer.

### Example 5

TABLE 2
the list of materials used in microencapsulation process of the probiotic
according to the present invention in this non-limiting Example

| Materials: | |
|---|---|
| L. Gasseri | Probiotic Bacteria |
| Maltodextrin | Substrate |
| Trehalose | Substrate |
| Cystein-HCl | Stabilizer- Antioxidant |
| Microcrystalline cellulose (MCC) | Glidant |
| Polyethylene glycol 1000 | Binder |
| Polyethylene glycol | PCM |

Polyethylene Glycol with different molecular weights was used as PCM for stratifying probiotic core granules. The molecular weight and the melting points of this series of PEG used in this experiment have been summarized in Table 3.

First a mixture of trehalose (80 g), probiotic bacteria L. Gasseri 57C (Biomed) (60 g), cystein-HCl (3 g) and maltodextrin (157 g) was loaded into Innojet Ventilus (Innojet IEV2.5 V2). Then PEG 1000 (115 g) was melted at 50° C. and microcrystalline cellulose (MCC PH 105) (11.5 g) was added into the melted PEG and homogenized to obtain a uniform dispersion. Then the resulting homogenous dispersion was sprayed onto the above dry mixture under an inert atmosphere using nitrogen. The temperatures of pump head, liquid, and spray pressure were set at room temperature. By theses means granulates were obtained based on a melt granulation. The resulting granules were then coated by homogenous dispersion of PEG 1000 melt (30 g) and MCC PH 105 (3 g) under an inert atmosphere using nitrogen to obtain granules coated by the first PCM coating layer. PEG 2000 was melted and then MCC PH 105 (10% w/w MCC/PEG) was added and homogenized to obtain a homogenous dispersion. The latter homogeneous dispersion was then sprayed onto the above granules coated by the second PCM coating layer to reach 10% weight gain thus obtaining granules coated by the third PCM coating layer. The resulting granules coated by the third PCM coating layer was discharged and kept in freezer for 2 hours. Then frozen granules coated by the third PCM coating layer were loaded again into Innojet Ventilus (Innojet IEV2.5 V2). PEG 4000 was melted and homogenized to obtain a homogenous dispersion. The latter homogeneous dispersion was then sprayed onto the above granules coated by the third PCM coating layer to reach 10% weight gain thus obtaining granules coated by the fourth PCM coating layer. PEG 6000 was melted and MCC PH 105 (10% w/w MCC/PEG) was added and homogenized to obtain a homogenous dispersion. The latter homogeneous dispersion was then sprayed onto the above granules coated by the fourth PCM coating layer to reach 20% weight gain thus obtaining granules coated by the fifth PCM coating layer. Then a solution of hydroxypropyl cellulose (HPC) in water (7% w/w) was prepared and sprayed onto the above resulting granules coated by the fifth PCM coating layer to reach 10% weight gain (w/w).

TABLE 3
molecular weight and melting point of different PEGs used
as PCM in Example 1 according to the present invention

| | | Molecular | Melting |
|---|---|---|---|
| | | Weight | point |
| Polymer | Function | (KD) | (° C.) |
| Polyethylene | First PCM | 950-1050 | 35~40 |
| glycol 1000 (PEG 1000) | | | |
| Polyethylene | Second PCM | 1400-1600 | 44-48 |
| glycol 1500 (PEG 1500) | | | |
| Polyethylene | Third PCM | 1800-2200 | 48~52 |
| glycol 2000 (PEG 2000) | | | |
| Polyethylene | Fourth PCM | 3700-4400 | 53~58 |
| glycol 4000 (PEG 4000) | | | |
| Polyethylene | Fifth PCM | 5600-6600 | 55~60 |
| glycol 6000 (PEG 6000) | (outermost) | | |

While the invention has been described with respect to a limited number of embodiments, it will be appreciated that many variations, modifications and other applications of the invention may be made.

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. It will also be appreciated by persons skilled in the art that the present invention is not limited by what has been particularly shown and described hereinabove.

1. A layered composition for a sensitive active material, comprising a core for containing the active material and a plurality of layers surrounding said core, each layer being temperature specific and each layer comprising one or more materials that are suitable for ingestion.

2. The composition of claim 1, wherein said active material is probiotic bacteria.

3. The composition of claim 1, wherein said active material is a pharmaceutically active material.

4. The composition of claim 3, wherein said pharmaceutically active material is sensitive to humidity and/or temperature.

5. The composition of claim 1, wherein said active material is a nutraceutically active material.

6. The composition of claim 5, wherein said nutraceutically active material is at least one of omega 3 fatty acids, omega 6 fatty acids, omega 7 fatty acids, omega 9 fatty acids or a combination thereof.

7. The composition of claim 1, wherein said plurality of layers surrounding said core are separated from each other by at least one soluble polymer.

8. The composition of claim 1, wherein said layered composition further comprises an outermost coating layer which is preferably soluble in the GI tract.

9. The composition of claim 1, wherein at least one layer completely surrounds said core.

10. The composition of claim 1, wherein at least one layer only partially surrounds said core.

11. The composition of claim 1, wherein said plurality of layers comprises a core layer for at least partially surrounding said core and at least one additional layer for at least partially surrounding said core layer, wherein each layer comprises a polymer having a melting point and wherein a melting point of said polymer of said core layer is the lowest of all melting points of all layers and wherein a melting point of said polymer of said at least one additional layer is higher than said melting point of said core layer.

12. The composition of claim 11, wherein a melting point of a polymer of each additional layer is higher than a melting point of a polymer of a preceding layer, in order of application of said layers.

13. The composition of claim 12, wherein each layer comprises a polymer having a phase change property such that said polymer absorbs heat from a surrounding environment with a low change in temperature or with no change in temperature.

14. The composition of claim 13, wherein said layers comprise a first coating layer which is said core layer and is adjacent to said core, comprising at least one first phase change material (PCM) having a melting point lower than 60° C. and higher than 20° C.; a second coating layer comprising at least one second phase change material (PCM) having a melting point lower than 60° C. and higher than 20° C., for at least partially coating the core coated with the first coating layer, wherein the second PCM has a melting point which is higher than the first PCM.

15. The composition of claim 14, wherein said PCM of said first layer has a melting point lower than 55° C. and higher than 20° C. and wherein said PCM of said second layer has a melting point lower than 55° C. and higher than 20° C.

16. The composition of claim 15, wherein said PCM of said first layer has a melting point lower than 50° C. and higher than 20° C. and wherein said PCM of said second layer has a melting point lower than 50° C. and higher than 20° C.

17. The composition of claim 16, further comprising a third coating layer comprising at least one third phase change material (PCM) having a melting point lower than 60° C. and higher than 20° C., for at least partially coating over second coating layer, wherein the third PCM has a melting point which is higher than the second PCM. The composition of claim 16, wherein said third PCM has a melting point lower than 55° C. and higher than 20° C.

18. The composition of claim 17, wherein said third PCM has a melting point lower than 50° C. and higher than 20° C.

19. The composition of claim 18, wherein each PCM in each layer has a different molecular weight.

20. The composition of claim 18, wherein said core comprises a stabilizer and at least one binder.

21-36. (canceled)
